(19) United States
(12) Patent Application Publication
(10) Pub. No.: US 2009/0047691 A1
(43) Pub. Date: Feb. 19, 2009

(54) Method for detecting a microorganism in a liquid sample

(75) Inventors: Alexander Karl Huwig, Schwetzingen (DE); Cornelia Weigand, Wasserburg (DE); Maja Borger, Schwetzingen (DE); Reto Grimm, Sargans (CH)

Correspondence Address: NIXON PEABODY LLP - PATENT GROUP, 1100 CLINTON SQUARE, ROCHESTER, NY, 14604, US

(73) Assignee: Ivoclar Vivadent AG, Schaan (LI)

(21) Appl. No.: 12/073,353

(22) Filed: Mar. 4, 2008

(30) Foreign Application Priority Data
Mar. 19, 2007 (EP) 07104443.2

(51) Int. Cl. G01N 33/569; C12M 1/00

(52) U.S. Cl. 435/7.34; 435/287.2; 435/7.2

(57) ABSTRACT

A method for the detection of a microorganism in a liquid sample, and in particular a method for the quantitative or semi-quantitative detection of a cariogenic microorganism such as a certain bacterium, for example in a saliva sample is described. Moreover, a test strip and test system with at least two test strips is described which are suitable for use in the described detection methods. Also, kits for carrying out detection methods according to the invention are also described.

This application claims priority pursuant to 35 U.S.C. § 119, to European Patent Application No. 07104443.2 filed Mar. 19, 2007, the entire contents of which are incorporated herein by reference.

### FIELD

The present invention relates to a method for the detection of a microorganism in a liquid sample. The invention also relates in particular to a method for the quantitative or semi-quantitative detection of a cariogenic microorganism, such as a specific bacterium, for example, in a saliva sample. Moreover, the present invention relates to a test strip and test system with at least two test strips is described which are suitable for use in the detection methods described. Finally, the invention relates to kits for carrying out the detection method according to the invention.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art.

The detection of microorganisms in biological samples, e.g., in body fluids of a patient is the aim of numerous diagnostic methods described in the state of the art. Pathogen-specific detection can be achieved in numerous different ways. Thus numerous infections caused by pathogenic microorganisms are diagnosed in human and veterinary medical practice via the detection of pathogen-specific nucleic acid sequences (DNA or RNA).

In the case of numerous diseases it is helpful to be able to effect an approximate assessment of the concentration of a certain microorganism in a sample from the patient, in order to be able to prognosticate the further development of the disease. In other cases, the determination of the concentration of certain microorganisms in a sample obtained from the patient permits the assessment of the risk of developing a disease.

The risk of developing dental caries, for example, has been shown to be associated with an increased occurrence of certain germs of the oral flora (e.g., bacteria of the genus Streptococcus and Lactobacillus). For this reason, test procedures have been developed which provide for an examination of saliva samples with a view to the concentration of these germs by plating out the samples on selective nutrient media. The cultivation of samples on nutrient media, however, is relatively time-consuming and requires the cultivation of Streptococcus and Lactobacillus and counting out of colonies of these organisms. An evaluation of the results can typically take place only about one to three days or later after plating out of the cells.

Alternative methods for the quantitative or semi-quantitative determination of certain microorganisms in a sample are based on the amplification of sequences which are specific for the microorganism to be detected (e.g., quantitative PCR or RT-PCR). In these processes, DNA purification is usually first carried out starting out from a sample (for example, blood, serum, tissue or similar) from a patient. The purified DNA can subsequently be employed in the amplification process concerned, primers being used, with their sequence being specific for the pathogen to be detected. The quantification is usually carried out at the end of the procedure, the quantity of PCR product providing information on the original quantity of DNA present in the sample. This can in turn be correlated to the number of cells originally present in the sample.

The methods for the detection of microorganisms based on the amplification of nucleic acids, however, have the disadvantage that relatively complex equipment such as PCR thermocyclers, are required for their execution. Moreover, further processes are regularly necessary in order to evaluate the results of the PCR procedure, e.g., electrophoretic separation processes. This means that conclusions regarding the presence of a microorganism in the sample examined can be obtained only after a considerable time delay.

### SUMMARY

It is consequently an object of the present invention to provide devices and methods by which the presence of certain microorganisms, preferably pathogenic microorganisms, in a liquid sample can be detected reliably and reproducibly. The methods should be suitable for carrying out without complex equipment and the result should become available, if possible, within 24 hours, 6 hours, or within 1 hour. The devices and methods should, moreover, allow a quantitative and/or semi-quantitative assessment of a sample examined.

The present invention provides in a first aspect a test strip for the quantitative or semi-quantitative detection of a microorganism in a liquid sample, comprising:
a) a first area for accepting the liquid sample, the first area having a labelled antibody which is capable of reacting with the microorganism to be detected by forming an immunocomplex;
b) a second area spaced from the first area, having a capture antibody which is capable of reacting with the microorganism to be detected by forming an immunocomplex and which is immobilised on the test strip;
   wherein the test strip is designed in a manner that the labelled antibody, on contact of the first area of the test strip with a mobile solvent, moves into the second area of the test strip such that in the presence of the microorganism to be detected in the liquid sample which is applied in the first area, an immunocomplex is formed from the labelled antibody, the capture antibody and the microorganism which complex can be detected by labelling of the labelled antibody in the second area of the test strip, and wherein the intensity of the signal provided by the labelling provides information on the concentration of the microorganism in the liquid sample.

According to a further aspect, the present invention provides a method for the quantitative or semi-quantitative detection of a microorganism in a liquid sample, comprising applying the liquid sample onto the test strip described above, contacting the test strip with a mobile solvent, incubating the test strip under conditions in which the labelled antibody moves, on contact of the first area of the test strip with a mobile solvent, into the second area of the test strip, and detecting the signal in the second area of the test strip; wherein the intensity of the signal provided by the labelling provides information on the concentration of the microorganism in the liquid sample.

According to yet another aspect, the present invention provides a test system comprising two test strips according of the type described above and a housing comprising two pairs of apertures, each pair comprising a first aperture and a second aperture spaced from the first aperture, and an inlet aperture for the two test strips, wherein the inlet aperture and the two pairs of apertures are arranged such that the two test strips can be inserted through the inlet aperture into the housing in such a manner that two spaced areas of the test strips are accessible through the first apertures, and the second apertures.

### BRIEF DESCRIPTION OF THE DRAWING FIGURE

FIG. 1 is a schematic illustration of a test system constructed according to one embodiment of the present invention.

### DETAILED DESCRIPTION

The term “test strip”, as used here, refers to an elongate matrix or membrane which preferably consists of a material which permits a lateral flow, mediated by capillary forces, of a liquid or a mobile solvent through the matrix or membrane. Suitable materials which permit such a chromatographic separation of different substances in a liquid sample, comprise, for example, cellulose or nitrocellulose, glass fiber, nylon, paper, cotton or materials of the test strip type of glass, silicone, plastic and metal into which suitable channels have been introduced. Suitable test strips are commercially available from different manufacturers in different forms (e.g., the Prima 40, 60 or 80 membranes from Schleicher & Schuell).

A test strip according to the invention may comprise a labelled antibody which, alone or in the form of an antigen-antibody complex with the microorganisms to be detected, is able to move in a liquid stream essentially unhindered through the pores of the test strip. In addition, the test strip comprises an adjacent area in the direction of flow with an immobilised capture antibody (i.e., an antibody which is fixed on the test strip such that, on contact with a mobile solvent no movement of the antibody is possible through the test strip). As used herein, a mobile solvent refers to a liquid which is capable of transporting the labelled antibody from a first area of the test strip into a second area of the test strip if it is brought into contact with the test strip at the end of the test strip facing the first area. Preferably, the test strip is immersed into the mobile solvent by this end. Conventional buffers are used as mobile solvents which do not interfere with the antibody reaction. The type of labelling of the antibody movable in the membrane also plays a part regarding the composition of the mobile solvent. Insofar as the antibody is labelled with an enzyme, for example, whose activity is to be detected, the mobile solvent must not interfere with this enzyme activity. Suitable mobile solvents comprise, for example, buffered salt solutions and the like. In addition, the mobile solvent may also contain protein stabilising reagents such as serum albumin, carbohydrates such as sucrose, or polyols such as sorbitol. Preservatives such as sodium azide, benzalkonium chloride or ProClin may also be added. Both the labelled antibody and the capture antibody are selected for a microorganism to be detected. As used above, the term “microorganism” refers to both prokaryotic as well as eukaryontic single cell organisms such as bacteria, yeasts or protozoae. In addition, the term microorganism also includes viruses, in particular viruses relevant in human or veterinary medicine. According to one embodiment of the present invention, the microorganism to be detected can be one or more bacteria.

The microorganisms to be detected by means of a method according to the invention are pathogenic, or optionally pathogenic bacteria which populate the oral cavity of mammals, such as humans, particularly bacteria which are associated with diseases of the teeth or the gums. According to one embodiment, the labelled antibody and/or the capture antibody is directed to bacteria of the genus Streptococcus, Lactobacillus, Actinomyces, Prevotella, Actinobacillus, Porphyromonas, Tannerella, Treponema, Fusobacterium, Enterococcus and Helicobacter, bacteria of the genus Streptococcus and Lactobacillus being particularly important in view of their relevance for the formation of caries.

The antibody can be directed against strains of Streptococcus mutans, Streptococcus sobrinus, Streptococcus sanguis, Streptococcus gordonii or Streptococcus mitis. Of these representatives of the genus Streptococcus it is known that they play a part, as initial occupants of the tooth surface, in the formation of caries. According to a further preferred embodiment, the microorganism which is to be detected by the method and test system according to the invention consists of strains of Lactobacillus acidophilus, Lactobacillus gasseri, Lactobacillus casei or Lactobacillus rhamnosus (Caufield et al. (2007), caries Res. 41:2-8). Bacteria of the genus Lactobacillus are capable of converting sugar to acid by fermentation and are thus capable of promoting the demineralisation of the tooth enamel. Further cariogenic microorganisms comprise Actinomyces naeslundii and the yeast Candida albicans.

Bacterial organisms such as Actinobacillus actinomycetemcomitans, Porphyromonas gingivalis, Tannerella forsythensis, Treponema denticola, Fusobacterium nucleatum and Prevotella intermedia which take part in the development of periodontitis and peri-implantitis (Mombelli (1997), Curr Opin Periodontol, 4:127-36) can be detected by the method according to the invention. Further microorganisms suitable for this method of detection comprise those which cause halitosis, such as Streptococcus salivarius (Sterer and Rosenberg (2006), J Dent Res, 85:910-914), microorganisms which produce volatile sulphur-containing compounds (Krespi et al. (2006), Otolaryngol Head Neck Surg, 135:671-676) and microorganisms which produce beta-galactosidase (Stereret al. (2002), J Dent Res, 81:182-5), or those which infect the dental root such as Treponema denticola, Enterococcus faecalis and others (Foschi et al. (2006), J Dent Res, 85:761-765). Moreover, microorganisms can be detected in the oral cavity which indicate a risk of a disease occurring at another site in the body. Such organisms comprise, for example, Helicobacter pylori (Foschi et al. (2006), J Dent Res, 85:761-765; Riggio and Lennon (1999), J Med Microbiol, 48:317-322).

It is also possible to provide test strips with antibodies which are directed against yeast, in particular pathogenic yeast. Such yeasts may, for example, be yeasts of the genus Candida, Cryptococcus, Malassezia and Blastoschizomyces. Particularly, the antibodies of the test strip can be directed against a yeast selected from the group consisting of Candida albicans, Candida krusei, Candida tropicalis, Candida glabrata, Candida lusitaniae, Candida parapsilosis, Cryptococcus neoformans, Blastoschizomyces capitatus.

The test strip disclosed according to the invention, as well as the method of detection and test systems making use thereof, are suitable in particular for use in the examination of saliva for cariogenic (i.e., caries causing or promoting) germs in the oral cavity of a mammal, in particular a human being. The oral cavity of humans is normally colonized by more than 800 different germs which are present in a natural balance. This microflora which consists essentially of bacteria, however, also comprises organisms which are capable of damaging the health of the teeth. This relates in particular to bacteria which are capable of metabolising carbohydrates from food into organic acid. The acid, e.g., lactic acid, attacks the tooth enamel leading initially to demineralisation of the surface of the tooth. By continual colonization of the demineralised sites, lesions are thus formed in the course of time which are capable of attacking the dentine and the dental pulp through the tooth enamel.

The bacteria of the genus Streptococcus play a particularly important part in the formation of caries. The Streptococcus mutans and Streptococcus sobrinus species, in particular, are among the first microorganisms which newly colonize a cleaned tooth surface. Both species are capable of forming a polymeric mucus from food residues and saliva components by means of which they are capable of adhering to the tooth surface by forming a biofilm thereon. The biofilm formed by the Streptococci as the early colonizers is also referred to as plaque. It consists of a structured coating of living and dead microorganisms in a matrix of polysaccharides and glycoproteins. Plaque deposits itself particularly easily in the indentations of the chewing surface (fissures), in the area between the teeth (interdental cavity) and at the edge of the gum. The biofilm makes it also possible for other bacteria to settle on the surface of the tooth. These include in particular bacteria of the genus Lactobacillus such as Lactobacillus acidophilus. Bacteria of the genus Lactobacillus are capable of mixed acid fermentation and they are responsible for a large part of the acid formed in the mouth by microorganisms. Strains of the lactobacillus genus can always be identified as an active caries site. For the above-mentioned reasons, the detection of bacteria of the genus Streptococcus and Lactobacillus in the saliva and the plaque forms the focus of the present prevention-oriented practice and research.

According to one embodiment of the invention, a method is provided for determining the risk of caries in which the number of cells of Streptococcus mutans (or S. sorbrinus) and at least one bacterium of the genus Lactobacillus (hereafter called Lactobacillus sp.) is determined quantitatively or semi-quantitatively, wherein exceeding a defined limit value is indicative of an increased risk of caries disease. In the case of adults, for example, a count of 10⁴ colony forming units (cfu) per ml of so-called “mutans Streptococci” (this term summarizes Streptococcus mutans and Streptococcus sobrinus) and Lactobacilli indicates an increased risk of caries. With a germ count of more than 10⁶ cfu/ml, there is a considerably greater risk potential. Values approximately 10 times lower apply to children. This means that the method of the present invention should preferably have a sensitivity which allows the detection of 1000 cells/ml. With a sample volume of 10 to 100 ml, this corresponds to an accuracy of 10 to 100 cells. Such a sensitivity is obtained, for example, upon labelling of the antibody with colloidal gold or colloidal silver.

Antibodies for the detection of pathogen microorganisms have been adequately described in the state of the art and are marketed by numerous commercial suppliers. In addition, antibodies can be produced against a specific microorganism by means of known methods. The production of suitable antibodies to microorganisms can take place starting out from molecular structures such as proteins or carbohydrates, which are sufficiently specific for the organism to be detected. This means that the antibodies used recognise the microorganism to be detected on the basis of certain molecular structures and are capable of binding to the same. In principle, any protein or carbohydrate which sufficiently specifies the microorganism to be detected can be used in order to produce suitable monoclonal or polyclonal antibodies to epitopes of the protein, using the above-mentioned processes. Particularly, the molecules acting as antigens are proteins of the microorganism, e.g., cell wall proteins. They can be obtained by culturing and disrupting the cells of the corresponding microorganisms. The cells are separated from the culture medium according to conventional processes, washed in buffered solution (e.g., in phosphate buffered saline) and disrupted. For the cell disruption, any desired method known in the state of the art can be used (Pingoud and Urbanke, 1997, Arbeitsmethoden der Biochemie (Working methods of biochemistry), Walter de Gruyter Verlag, Berlin, page 45-52). Subsequently, the desired cell wall components are purified by further purification processes e.g., dialysis, chromatography, filtration etc and, if necessary, lyophilised for prolonged storage.

According to an exemplary embodiment, the molecule which is used for producing antibodies is a protein, occurring naturally in the microorganism, of complete length or a variant or a fragment of such a protein. Variants of a protein or polypeptide should be understood to mean those proteins or polypeptides which differ by one or several exchanges of amino acids from the amino acid sequence of the original protein or polypeptide. The amino acid exchange can be a conservative or non-conservative amino acid exchange. Basically, any amino acid residue of the amino acid sequence of the original protein or polypeptide can be exchanged by another amino acid as long as the exchange does not lead to a major change in the structure of the epitope or the epitopes of the protein or polypeptide. In general, variants of a protein or a polypeptide may exhibit a significant agreement with the original protein or polypeptide. Preferably, the amino acid identity amounts to more than 60%, 70%, 80%, 90% or more than 95%.

Those proteins or polypeptides which differ from the original protein or polypeptide by one or several additional amino acids are also considered to be variants. These additional amino acids may be present within the amino acid sequence of the original protein or polypeptide (insertion) or they may be added to one or both termini of the protein or polypeptide. Basically, insertions can take place at any position provided the exchange does not lead to a major change in the structure of the epitope or the epitopes of the protein or polypeptide. Variants are also contemplated in which one amino acid or several additional amino acids are added to the C terminus and/or N terminus. Consequently, the term variants also comprise fusion polypeptides in the case of which the original protein or polypeptide is fused with flanking sequences which permit purification of the protein in the case of heterologous expression. Examples of such sequences comprise histidine modules such as the 6×His-tag, which permit purification of the fusion polypeptide via the affinity to immobilised nickel ions, or domains of protein A, a bacterial cell wall protein of Staphylococcus aureus with a specific activity towards the Fc-region of immunoglobulines of class G (IgG). Other flanking sequences which can be used for purifying fusion polypeptides are sufficiently known to the skilled person. Moreover, the term “variants” also comprises those proteins or polypeptides in the case of which, in comparison with the original protein or polypeptide, one or several amino acids are lacking. Such deletions may affect any amino acid position provided the exchange does not lead to a major change in the structure of the epitope or the epitopes of the protein or polypeptide.

The present invention also comprises the use of immunologically active fragments of the original protein or polypeptide obtained from the microorganism and its variants as defined above for the production of the antibodies used according to the invention. Suitable fragments are considered to be, within the scope of the present invention, those peptides or polypeptides which differ from the original protein or polypeptide and its variants by the lack of one or several amino acids at the N terminus and/or the C terminus of the peptide or polypeptide, wherein at least part of the immunological activity, i.e., its antigenic properties, is retained.

Derivatives of the original protein or polypeptide obtained from microorganisms, or its variants, may also be used according to the invention in order to produce the necessary antibodies. Derivatives refer to proteins or polypeptides, which are present and exhibit structural modifications in comparison with the original protein or polypeptide or its variants, such as modified amino acids. According to the invention, these modified amino acids may be amino acids which have been modified either by natural processes such as processing or post-translational modifications or by chemical modification processes sufficiently known in the state of the art. Typical modifications to which the amino acids of the polypeptide may be subjected comprise phosphorylation, glycosylation, acetylation, acylation, branching, ADPribosylation, crosslinking, disulfide bridge formation, formylation, hydroxylation, carboxylation, methylation, demethylation, amidation, cyclisation and/or covalent or non-covalent bonding to phosphotidyl inositol, flavine derivatives, lipoteichonic acids, fatty acids or lipids. Such modifications have been described in the relevant literature, e.g., in Proteins: Structure and Molecular Properties, T. Creighton, 2^{nd} edition, W. H. Freeman and Company, New York (1993).

A test strip constructed according to embodiments of the invention comprises a first area which serves the purpose of taking up the liquid sample (in the present case also referred to as sample acceptance area). In this area, the test strip contains a labelled antibody which provides a detectable signal. The antibody is applied onto the matrix such that it migrates, on contact of the test strip with a mobile solvent, in the direction of flow which is predetermined by the capillary forces, through the membrane in the direction of the subsequent detection zone. This means that the labelled antibody may be applied onto the test strip only, such that it is reversibly held on the test strip and can be mobilised by the mobile phase buffer or the sample volume. As soon as the labelled antibody is in fluid contact with the mobile solvent, it detaches itself from the matrix and is transported by the mobile solvent through the matrix. Insofar as the liquid sample which has been applied onto the sample acceptance area of the test strip contains the microorganism to be detected, the freely moveable labelled antibody reacts with this microorganism. This means that the labelled antibody recognises an antigenic structure of the microorganism and binds to it (e.g., to a surface protein of a bacterium). In this case, an antigen-antibody complex is formed between the labelled antibody and the antigenic structure and transported through the porous structure of the test strip. The antibody or the immunocomplexes subsequently move(s) to a second area of the test strip which is situated downstream from the first area in the direction of flow. In this second area (also referred to as the detection zone), the test strip comprises an immobilised capture antibody which is also directed against the microorganism to be detected. This means that the second antibody (capture antibody) cannot move freely through the test strip but serves for “capturing” the antigen originating from the microorganism. Insofar as the labelled antibody has not bound any microorganism (because the microorganisms to be detected is present in the sample either not at all or in only minor quantities), the labelled antibody is able to pass through the detection zone unhindered. Immunocomplexes of the labelled antibody and microorganism, on the other hand, are recognised and bound by the immobilised antibody which exhibits a specificity for the microorganism such that a type of “sandwich complex” is formed from the two antibodies and the microorganism. The formation of such a complex leads to an enrichment of the labelled first antibody in the detection zone formed by the capture antibody. Here, the evaluation of the test takes place by a suitable detection of the labelling of the first antibody. The strength or intensity of the signal provided by the labelling provides information on the quantity of the microorganism present on the test strip. It is preferred to apply the capture antibody in the form of a thin line onto the test strip such that the “sandwich complexes” form a sharply defined band on the test strip. Such bands simplify the reading of the signal strength. According to a further embodiment of the invention, a single test strip may comprise more than one pair of labelled antibody and capture antibody, so that different microorganisms may be detected on the same test strip, for example, 2, 3, 4, or 5 different microorganisms.

The test strips according to the invention may additionally comprise a control area which follows the detection zone in the direction of flow of the mobile solvent and provides information on whether the test strip concerned is basically capable of functioning. The control area of the test strip may comprise an immobilised control antibody, for example, which binds a mobile labelled control antibody applied in the sample acceptance area, the mobile labelled control antibody reacting neither with the immobilised capture antibody in the detection zone nor with the microorganism to be detected. Preferably, the labelled control antibody is labelled in the same way as the labelled antibody which is directed against the microorganism. A further embodiment provides for an antibody to be used as immobilised control antibody which binds the labelled antibody directed against the microorganism to be detected. This immobilised control antibody may be an anti-species antibody. For example, a monoclonal antibody from a mouse (such as SWLA1) is used as labelled antibody for binding to the microorganism to be detected, the immobilised control antibody may be a conventional anti-mouse antibody. Only if a positive signal is obtained in the control area for the second labelled antibody can the absence of a signal in the detection zone of the test strip be interpreted as meaning that the microorganism to be detected is not contained in the sample or is contained in a concentration which is below the limit of detection.

An improvement in the test sensitivity can be achieved by doping the test strips according to further embodiments of the invention. In this case, so many cells of the microorganism to be detected are applied in the sample acceptance area that a concentration of cells below the limit of detection is obtained. If the limit of detection for a specific microorganism, for example, is 10³ cells per ml, from 100 cells per ml (10% of the limit of the detection) up to 900 cells per ml (90% of the limit of detection) can be added to the sample acceptance area of the test strip. With an addition of 90% of the limit of detection, the sensitivity for the microorganism concerned is improved to 10² cells per ml since the test strip then already comprises 90% of the cells necessary for a detection operation. Obviously, parts of the microorganisms to be detected can also be used for doping insofar as these are recognised by the corresponding antibodies of the test strip. In this case, antigenic proteins or fragments thereof, for example, may be involved. Short peptides can also be used which comprise the epitope for the antibody to be used for detection. According to the invention, those test strips are preferred in particular which have been doped with cells of Streptococcus, in particular Streptococcus mutans or Streptococcus sobrinus. The test strips may comprise 70%, 80%, 90% or 95% of the cells necessary for a detection operation. The actual limit of detection depends on the design of the test system and in particular the labelling of the antibody and can be established for any system by corresponding tests. According to a specific embodiment, the first area of the test strip is doped with the microorganism to be detected or immunologically active parts thereof. The first area may comprise 90% of the quantity of the microorganism to be detected necessary for detection.

Applying the antibody onto the test strips can be effected by any conventional dispensing method. For this purpose, the antibody solution concerned is applied onto the non-woven materials or membranes by means of a cannula. The width of the test line can be influenced in this case by selecting the diameter of the cannula. Dispensing takes place in such a way that the cannula is moved to and fro over the non-woven materials or membranes. The quantity applied in each case can be adjusted by the rate with which the cannula is moved as well as by the concentration of the reactants in the dispensing solution. Typical application rates for the conjugated labelled antibodies are in the region between 10-250 optical units of the stained colloids per 5 mm of non-woven material width. For the capture and control line antibodies, rates of between 0.01 and 0.5 μl/mm are obtained. In this connection, reference should be made to the IEMA-analogous test principle (immunoenzymetrically analogous test principles) which is described in EP-A-0 407 904, EP-A-0 353 570 or DE-OS 40 24 919, for example.

The antibodies used on the test strips according to the invention are antibodies which are directed against the microorganism to be detected. As used here, the term antibodies means immunoglobulines (Ig) and immunologically active parts thereof. The antibodies used within the scope of the invention for the detection of the microorganisms may be any type of antibody, e.g., polyclonal, monoclonal, chimeric, human, humanized, synthetic or anti-idiotypic antibodies. Moreover, fragments of the above-mentioned antibodies can also be used to detect the microorganisms concerned in the sample. Fusions proteins which comprise antibodies or their fragments and comprise the sequence of the antibodies fused to a further peptide or protein can be used within the scope of the invention.

The antibodies or their fragments can be obtained from any desired mammals, e.g., rabbits, rats, mice, goats, horses, primates or humans. Alternatively, the antibodies may be synthetic antibodies which can be produced, for example, by recombinant processes. On the basis of differences in the heavy chain, the antibodies induced in mammals against antigens are usually divided into different classes. In the case of most of the more highly developed mammals, there are five different classes of immunoglobulins which are referred to as IgG, IgA, IgM, IgD and IgE. In addition, there may be further sub-classes. These classes and sub-classes are equally suitable for carrying out the disclosed methods. However, it is preferred if the antibodies are those of class IgG, e.g., from the sub-classes IgG1, IgG2, IgG2a, IgG2b or IgG3.

According to a particular embodiment of the invention, the antibodies which are used within the scope of the invention for the detection of the pathogenic microorganisms are monoclonal antibodies. As used herein, “monoclonal antibodies” refers to an immunoglobulin which is produced by a cell line which is based on a single B lymphocyte. Monoclonal antibodies are directed against a single epitope in a specific antigen. They can be produced by methods adequately described in the state of the art. Monoclonal antibodies can, for example, be produced by using hybridoma cell lines such as those described by Köhler and Milstein, Nature, 256, 495-397, (1975) and in Harlow and Lane “Antibodies”: Laboratory Manual, Cold Spring, Harbor Laboratory (1988); Ausubel et al., (eds), 1998, Current Protocols in Molecular Biology, John Wiley & Sons, New York). The method is based on fusing a B cell producing antibodies with an immortalised cell line as a result of which a hybrid cell is produced which produces antibodies of a defined specificity to an unlimited extent. First of all, a specific antigen against which the monoclonal antibody is to be produced is injected in the usual way into a rodent, such as a mouse, a rabbit or a rat. A (if applicable derivated) protein or polypeptide or a suitable fragment thereof preferably acts as antigen in this case. By injecting the antigen, the production of antibodies against the antigen in the B lymphocytes of the immune system is stimulated.

The antibodies formed accumulate in the spleen and in the lymph nodes of the animal and are taken from there for fusion with cells of the immortalised cell lines. Immortalised cell lines are usually transformed cells from mammals, in particular myeloma cells of rodents, cattle or humans. Preferably, myeloma cells from the mouse are used. According to an alternative embodiment of the invention, the hybridoma cells originate from a human. Moreover, human-mouse hetero-myeloma cells have been described for the production of human monoclonal antibodies. The hybridoma cells formed combine the property of their origin cells. Like the B lymphocyte, they form a certain antibody species and additionally exhibit the properties of the myeloma cell of multiplying in vitro to an unlimited extent. The antibody molecules separated off by the hybridoma cells into the culture medium can subsequently be examined for the presence of the desired monoclonal antibody.

The clones with the best binding specificities with respect to the antigen, for example the protein, are subsequently detected by screening. The binding specificities of the antibodies produced by the hydridoma cells are usually examined by methods such as immunoprecipitation, radio immunoassay (RIA) or ELISA (Enzyme Linked Immunosorbent Assay). Such methods have been adequately described in the state of the art and require no further explanation herein. Suitable clones are stored and the supernatant of these clones is harvested as required.

On cultivation in roll flasks of 5 l, hybridoma cells can lead to a yield of 20-70 mg of the desired monoclonal antibody. In addition, hybridoma cells can be used for the production of antibodies on a large scale of 200-600 mg by immunising animals such as mice directly with these hybridoma cells and collecting the antibodies formed in the course of the subsequent infection in the abdominal cavity fluid (ascites) (Leenaars and Hendriksen (2205), ILAR J, 46:269-279; Hendriksen and de Leeuw (1998), Res Immunol, 149:535-542).

Apart from methods based on the production of hydridoma cells, other methods have been described for the production of monoclonal antibodies. These methods are equally suitable for the production of the antibodies used within the scope of the invention. It is, for example, possible to produce monoclonal antibodies by recombinant DNA processes as described e.g., in U.S. Pat. No. 4,816,567. Monoclonal antibodies may additionally be isolated from an antibody phage library such as described by Clackson et al. (1991), Nature, 352:624-628.

Insofar as the test strip according to the invention is formed for detecting Streptococcus mutans, the labelled antibody and/or capture antibody of the membrane may be, for example, the monoclonal antibody SWLA1 (ATCC HB12559), SWLA2 (ATCC HB12560) or SWLA3 (ATCC HB12558), which are described in WO 00/11037. The monoclonal antibody SWLA1 which has been selected on the basis of a high selectivity to Streptococcus mutans, is an immunoglobulin of the IgG type which is conjugated for the test with colloidal silver or gold of a particle size of 20 to 300 nm, preferably 20 to 80 nm and particularly preferably 40 nm (compare also Shi et al. (1998) Hybridoma, 17:365-371; Gu et al. (2002), Hydrid Hybridomics, 21:225-232). Insofar as the test strip according to the invention is formed for detecting bacteria of the genus Lactobacillus, the labelled antibody and/or the capture antibody of the membrane may be, for example, the monoclonal antibody SWLA5 of type IgM (Gu et al. (2002) Hybrid Hybridomics, 21:469-478). For the detection of Lactobacilli, no specificity with respect to the type or strain is required, i.e., all antibodies sufficiently specific for bacteria of the genus Lactobacillus are suitable for use in the methods and test systems of the present invention.

Within the scope of the present invention, polyclonal antibodies can also be used in connection with the methods and test systems described. Polyclonal antibodies are obtained from the sera of animals which have been immunised with an antigen (e.g., a certain protein). Polyclonal antibodies are, in the end, a heterogeneous mixture of antibodies which originate from more than one B lymphocyte and are frequently directed against different epitopes of a specific antigen. The term “polyclonal antibody” as used herein, comprises also a mono-specific antibody which is obtained after purifying the different antibodies obtained from the sera (e.g., coupled by means of a column to the peptides, which contain a specific epitope).

During the production of polyclonal antibodies, a host animal (e.g., a rabbit, a goat, a mouse or any other mammal) is first immunised by one or several injections of the antigen, e.g., the immunogenic protein (or a fragment or a variant thereof). This means that the antigen is injected into these animals once or several times. In the immunogenic composition, a naturally occurring, native protein, a chemically synthesised polypeptide which represents this protein or a part thereof or a recombinantly express protein or a fragment thereof can be used. The protein may, moreover, be coupled to a further protein which is known to have an immunising effect in the mammal used as host. Such accessory proteins are well known in the state of art and comprise, for example, the so-called keyhole limpet hemocyanin, serum albumin, soy bean trypsin inhibitor and others. The compositions used for immunisation generally also comprise an adjuvant. A substance is referred to herein as “adjuvant” which enhances the immunological reaction of the host. Such adjuvants are also known in the field of immunology and comprise Freund complete or incomplete adjuvant, for example, as well as aluminium hydroxide and others.

Antibodies used within the scope of the present method may be also chimeric antibodies. As used here, the term “chimeric antibodies” refers to an antibody which is composed of different components which originate from different mammal species. Chimeric antibodies are consequently “mixed” antibodies which consist of molecular building blocks from (at least) two different antibodies. Usually, the variable regions of a chimeric antibody are derived from a mammal species, whereas the constant regions originate from another species. Examples of chimeric antibodies comprise humanized antibodies, for example. Chimeric antibodies and methods for their production are well known to the skilled person working in the field and have, moreover, been described, for example, by Boulianne et al. (1984) Nature 312:643-646; Cabilly et al. (1984) Proc. Natl. Sci. USA 81:3273-3277 and Neuberger et al (1985), Nature 314:268-270. Principles and considerations which are relevant in the construction of chimeric antibodies can also be found in Harlow and Lane “Antibodies”: Laboratory Manual, Cold Spring, Harbor Laboratory (1988).

In addition, functional fragments of antibodies, apart from complete antibody molecules, can also be used for the methods and test systems according to the invention. As used herein, functional fragments should be understood to mean those parts of the antibodies, as defined above, which retain at least a part of the binding affinity of the complete antibody from which they are derived and are consequently capable of binding to the corresponding antigen (in the present case to the microorganism to be detected) with a sufficient specificity. Fragments of antibodies are usually produced by proteolytic cleavage, chemical synthesis or recombinant DNA processes. Examples of suitable antibody fragments comprise Fab, Fab′, F(ab′)₂ and Fv fragments as well as single strand antibodies with a bonding affinity for the microorganism to be detected. Fragments of the monoclonal antibodies SWLA1, SWLA2, SWLA3 or SWLA5 are particular examples.

According to the invention, the labelled antibody and the capture antibody can be the same antibody. However, this is not essential for as long as it is guaranteed that both the labelled antibody and the capture antibody bind with a sufficient specificity to the microorganism to be detected, for example, Streptococcus mutans or Lactobacillus sp. It is, for example, possible to use antibodies which are directed against different molecules, e.g., different cell wall proteins of the same microorganism. Moreover, glycerophosphates and ribit phosphates which are linked with other sugars as well as with D-Alanin, can be recognised, wherein these units can be in turn linked with membrane lipids. Fats can also serve as antigenic structures.

Moreover, antibodies can be used which are directed against the same molecule, though different epitope structures being recognised. According to a preferred embodiment, either the labelled antibody or the capture antibody is a monoclonal antibody. It is particularly preferred that both the labelled antibody and the capture antibody are monoclonal antibodies.

According to a particular embodiment of the invention, the labelled antibody directed against the microorganism to be detected is conjugated to particles which provide a calorimetric signal. Such particles are sufficiently well known in the state of the art and comprise, for example, particles of colloidal gold or colloidal silver. Colloidal gold is characterised by a deep red color which is easy to perceive visually. Colloidal silver, on the other hand, has a yellowish color in the diluted state and a brown color in the concentrated state. According to the invention, the particles have a size of approximately 10-100 nm, particle sizes of approximately 20-80 nm and in particular approximately 40 nm are also contemplated. Apart from colloidal gold or silver, the antibodies can also be coupled to stained latex particles. As an alternative, other molecules with which the skilled person is familiar can be used for labelling of the antibodies. Such markers comprise in particular fluorescent markers, chemiluminescent markers, biotin, avidin, streptavidin, chromogenic groups which form a visually recognisable stain during their hydrolysis, or enzymes. Preferred enzymes comprise phosphatases (e.g., alkaline phosphatase), peroxidases (e.g., horseradish peroxidase), beta-galactosidase. In addition, the antibodies which are applied onto the test strip may also be detected with secondary antibodies which specifically bind to the antibodies on the test strip. For purposes of detection, the secondary antibodies may be labelled with the corresponding molecules, such as the above-mentioned enzymes.

The liquid sample which comprises the organism to be detected can comprise a body fluid such as whole blood, plasma, serum, liquor, urine, joint puncture specimen, saliva or similar. Liquid homogenates of essentially solid materials such as cell material which has been obtained from smears or biopsies, are in the present case also regarded as liquid samples according to the meaning of the invention. Depending on the type and origin of the sample and/or the type of the microorganism to be detected, it may be necessary or meaningful to effect a concentration of the cell material before carrying out the method according to the invention. Such a concentration may be carried out using a filter of a suitable pore size. The cells remaining on the filter can subsequently be resuspended in a smaller volume of liquid.

Insofar as necessary the liquids used as samples can be treated with further agents in order to increase the accessibility of the microorganisms to be detected. In this way, the sensitivity of the test system concerned can usually be substantially increased. For this purpose, the sample to be investigated is mixed with substances which reduce an interaction of the organisms with other components of the sample or an interaction of the organisms with each other. These substances comprise chelating molecules such as EDTA, NTA, DTPA, HEDTA or citric acid which inhibit interactions of the microorganisms with cations such as Ca²⁺, Cu²⁺, Mg²⁺, Fe²⁺, Fe³⁺ or Co³⁺ by complexing of the cations. Moreover, the samples can be pre-treated with proteolytic enzymes before application onto the test strips. Proteolytically active enzymes are sufficiently well known to the skilled person. They comprise, for example, the enzymes pepsin, papain, pancreatin, trypsin, chymotrypsin, chymosin, renin, cathepsin B and D and such like. These proteases cause the cleavage and decomposition of proteins of the sample, such as saliva proteins (mucines) and of proteins which cause or support an aggregation between individual cells of the microorganism. Sugar-cleaving enzymes such as glucosidases, amylases, lysozym, hyaluronidases and similar can contribute to an improvement in the test sensitivity by acting on glycoproteins and/or carbohydrate components of the microbial cell walls. Chaotropic compounds and ions of the Hofineister series can also be used. They reduce the extent of non-specific interactions, such as ionic interactions, hydrogen bridge bonding, Coulomb interactions. SH-group modifying reagents such as dithiothreitol, mercaptoethanol, glutathion and others can be added to the sample to cleave protein sub-units which are bonded by disulphide bridges. In addition, solutions or substances such as NaOH, HCl or buffered solutions can also be added to the sample concerned which change the pH by changing the pH of the liquid sample, a change in their consistency can be achieved which is advantageous for the chromatographic separation on the test strips.

The sample can be transferred to the test strip by means of suitable instruments. Basically, all instruments which permit the application of a certain volume of the sample onto the test strip in a reproducible manner can be used. According to a particularly preferred embodiment of the invention, the sample transfer onto the test strip is effected in as simple a way as possible, such as by means of a pipette. Moreover, suitable brush-type applicators are described in the state of the art such as those produced by Microbrush (Grafton, Wis., USA) which permit a satisfactorily reproducible transfer of a defined sample volume such as a saliva volume. In addition, such an applicator provides the possibility of taking plaque samples from individual teeth and transferring them into a buffer, such as into the mobile buffer. In this way, it is possible to determine the risk of disease, for example, at a specific site. Moreover, the sampling and transfer of sulcus liquid, such as of sample material from a gum pocket, is possible. For reasons of reproducibility, each individual determination of a microorganism ought to be effected with a separate applicator. The volume applied onto the test strips depends on the type of the sample to be examined and the concentration, to be expected, of the microorganism contained therein and to be detected. Insofar as the sample is a saliva sample, for example, the sample volume generally is less than 500 μl, for example, 300 μl, 100 μl, 50 μl or 10 μl. The sample volume is influenced by the type of the antibody, the type of visualisation of the antigen-antibody reaction and by the materials used for manufacturing test strip and is adjusted, as a function of these factors, always at the lowest possible level.

The present invention additionally provides methods for the quantitative or semi-quantitative detection of a microorganism in a liquid sample, such methods may comprise:
a) applying the liquid sample onto a test strip as defined above,
b) contacting the test strip with a mobile solvent,
c) incubating the test strip under conditions in which the labelled antibody moves, on contact of the first area of the test strip with a mobile solvent, into the second area of the test strip, and
d) detecting the signal in the second area of the test strip,
   wherein the intensity of the signal provided by the labelling provides information on the concentration of the microorganism in the liquid sample.

Methods according to the invention provides initially for the liquid sample to be investigated to be applied onto the sample acceptance area of the test strip provided for this purpose. Insofar as the sample has a sufficiently low viscosity and a sufficiently large volume, the use of a mobile solvent is not required. It is of course also possible to introduce the sample to be examined into a suitable mobile solvent and to apply a correspondingly large volume onto the sample acceptance area of the test strip. Better results, however, are achieved by applying a relatively small sample volume of less than 500 μl onto the test strip and starting chromatography by immersing the end of the test strip facing the sample acceptance area into a corresponding mobile solvent. Subsequently, the test strip is incubated under conditions which permit spreading of the liquid sample from the first area of the test strip (i.e., the test acceptance area) into the adjacent second area of the test strip (i.e., the detection zone) provided by the capillary direction of flow. Insofar as binding of the labelled antibody in the detection zone of the test strip takes place, the signal provided by the marker can be detected in this area. The detection depends on the type of labelling. In a simple embodiment, the labelling which is applied onto the first antibody is colloidal gold or colloidal silver such that detection is possible by visual sight examination. Depending on the type of labelling, however, it may also be necessary to carry out an enzyme reaction with a luminescent substrate or a fluorescent labelling, for example, by means of a fluorescence microscope. The antigen-antibody reaction can be continually read by light or reflection measurement.

The method may comprise, as the final step, the comparison of the detected signal with one or several reference values in order to provide a quantitative or semi-quantitative statement regarding the microorganism to be detected. Preferably, a color standard is used, which indicates the semi-quantitative assessment of the number of microorganisms to be detected which are present in the sample. In order to be able to carry out the application of the test in as simple a manner as possible and independently of additional equipment, it is preferred for the signal intensity to be read by a comparison with an optical standard which reflects the signal intensity in a stepwise manner. This corresponds to a further simplification since the signal can be determined discontinuously in stages, i.e., semi-quantitatively. The gradations of the standard are produced by way of defined concentrations of the microorganisms to be detected. The optical standards can be used in particular in the case of labelling of the first antibody with colloidal gold or colloidal silver. Antibodies which are labelled with enzymes, too, permit optical standards to be set up. Preferably, the optical standards comprise several stages which differ from each other regarding the signal intensity, i.e., the depth of color. According to one embodiment, optical standards with four stages are utilized. The optical standards can be produced by dilution series in the case where samples with a known germ count are applied onto the test strips under test conditions and the signal strength is recorded for each sample as reference value for a specific number of the respective microorganism. Obviously, calibration curves can also be recorded which permit an allocation of a detected signal to a specific number of microorganisms.

An additional binary gradation may be effected by way to the control line of the test strip (if present). The intensity of the control line can be adjusted to a concentration to be determined for each microorganism, for example, 10⁵ cfu/ml, such that an additional binary evaluation is possible by way of this control line. In the case of a four stage standard, for example, the second highest concentration stage can be related to the control line, i.e., when the intensity of the test signal corresponds to the control line signal, the second highest concentration of the microorganism is present in the sample. The following intensity gradation is obtained as a function thereof:

| | | |
|---|---|---|
| Test signal | > | Control line: highest concentration |
| Test signal | = | Control line: second highest concentration |
| Test signal | < | Control line: second lowest concentration |
| Test signal | = | 0 or << control line: lowest concentration |

In order to avoid errors, an attempt should be made to obtain at least a weak signal for all investigations so that the assessment of the concentration can always take place on the basis of a signal.

For the assessment of the caries risk, 10⁵ cfu mutans Streptococci (Streptococcus mutans, Streptococcus sobrinus) or 10⁵ cfu Lactobacilli per ml of saliva sample, for example, correspond, in the case of an adult, to an average risk of suffering from caries. The control line can be adjusted to this value such that a stronger signal than the control line indicates a higher risk and a weaker signal than the control line a lower risk. This adjustment can be exploited when assessing the test strips in order to permit the user to effect an evaluation of the caries risk as being high or low. The exact gradation into one of at least four stages can then be effected by a comparison with an evaluation curve as an optical standard, for example. The lower limit determined by the sensitivity is 5×10³ cfu/ml. If no or only a weak signal appears on the test line, this corresponds to the stage of 0. If the signal is distinctly present but weaker than the control, for example, approximately 10⁴, the signal is assessed as belonging to stage one. Stage 2 arises if the test line and the control line are of equal strength. Stage 3, i.e., the highest of 4 stages arises if more than 5×10⁵ cfu/ml are present.

According to a particular embodiment of the present invention, the method for the quantitative or semi-quantitative detection of a microorganism in a liquid sample is a method for assessing the risk of cariogenesis. According to a further preferred embodiment, the germ count of Streptococcus mutans and/or Lactobacillus sp. is determined quantitatively or semi-quantitatively in this caries risk test. It is within the scope of the method of detection to determine, in parallel, at least two types of bacteria simultaneously in a sample such as in the same saliva sample. For this purpose, two test strips can be used simultaneously. The first test strip comprises an antibody directed against Streptococcus, for example, SWLA1, as a labelled antibody and/or as capture antibody. The second test strip comprises an antibody directed against the Lactobacillus, for example, SWLA5, as a labelled antibody and/or as capture antibody.

Finally, the present invention also relates to a kit for carrying out methods for the quantitative or semi-quantitative detection of a microorganism in a liquid sample. The kit according to the invention may comprise one or several of the test strips described above as well as, if necessary, one or several buffers, one or several mobile solvents, one or several labelling reagents and/or one or several detection reagents for the detection of the labelled antibody. In addition, the kit comprises instructions for performing the detection method according to the invention. Usually the kit also comprises suitable reference standards which permit an allocation of signal strength and concentration of the microorganism in the sample. In the case of a calorimetric labelling of the labelled antibody used for detection, such as colloidal gold, the reference standard is for example a color chart which permits a correlation between the color depth and the concentration of the microorganism.

According to a particular embodiment of the invention, a test device is made available for the parallel, simultaneous detection of two microorganisms, such as Streptococcus mutans and Lactobacillus sp., in a liquid sample. The test system comprises two of the test strips according to the invention as defined above and a housing which comprises two pairs of apertures, each pair consisting of a first aperture and a second aperture spaced from the first aperture, and an inlet aperture for the two test strips, the inlet aperture and the two pairs of the first aperture and the second aperture being arranged such that the two test strips can be inserted through the inlet aperture into the housing such that in each case two spaced apart areas of the test strips are accessible through the first apertures and the second apertures. In this respect, the sample acceptance area of the test strip is accessible through the first aperture whereas the detection zone is accessible through the second aperture. According to a particular embodiment of the invention, the test system may comprise more than two of the above-defined test strips and a housing that is capable of accepting more than two test strips. For example, a test system according to the invention may comprise 3 to 10 different test strips for simultaneous use. Accordingly, the housing of the test system may be capable of accepting 3 to 10 test strips.

The use of a test strip as described above or of a test system as described above for the determination of the risk of cariogenesis is also subject matter of the present invention.

The invention is explained in further detail in the following by way of embodiments with reference to the drawing.

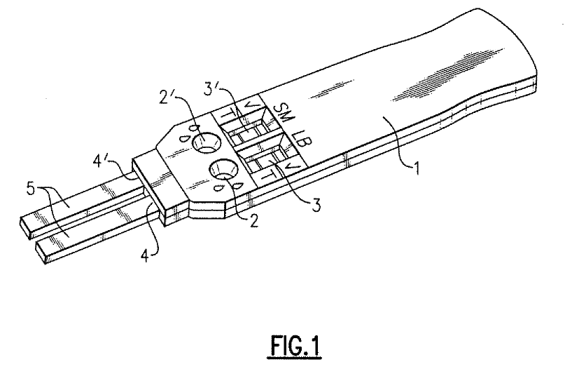

In FIG. 1, a test system according to the invention is depicted. The test system comprises a housing 1, which comprises a first, upper half-shell and a second lower half-shell. In the housing 1, two pairs each consisting of a first aperture 2, 2′ and a second aperture 3, 3′ are provided in the upper half-shell. In this regard, the first aperture 2, 2′ and the second aperture 3, 3′ of the two pairs are arranged spaced from each other, wherein in this embodiment preferred in this respect the connecting lines between the first aperture 2, 2′ and the second aperture 3, 3′ of the two pairs are extending parallel to each other.

Moreover, an inlet aperture is provided in the housing 1, which inlet aperture comprises, in this embodiment preferred in this respect, a first aperture portion 4, and a second aperture portion 4′. The inlet aperture portions 4, 4′ are arranged in the housing 1 such that two test strips 5 can be inserted through the aperture portions 4, 4′ of the inlet aperture into the housing 1 in such a manner that in each case two spaced apart areas of a test strip 5 are accessible through the first aperture 2, 2′ and the second aperture 3, 3′ of one pair of apertures. As explained below, the first apertures 2, 2′ serve as sample aperture and the second apertures 3, 3′ as windows for reading the antigen antibody reaction. This means that the areas of the test strip which are accessible through the first apertures (2, 2′) comprise the labeled antibody, whereas the areas of the test strips which are accessible through the second apertures (3, 3′) comprise the capture antibody and, if necessary, one or several control lines.

In order to carry out a measurement for the determination of the caries risk in a patient with the test system according to the invention, a saliva sample is first introduced into the sample aperture and/or the two first apertures 2, 2′ of the two pairs of apertures. Since the saliva sample generally has a consistency that is too high for initiating the chromatographic separation on the test strip, the two test strips 5 protruding from the inlet aperture portions 4, 4′ of the inlet aperture are immersed into a suitable mobile solvent which is subsequently drawn through the entire test strip 5 as a result of capillary forces.

Subsequently, the saliva introduced into the first apertures 2, 2′ is drawn from the region of the first apertures 2, 2′ into the region of the reading window and/or the second apertures 3, 3′, and carries out the reactions already described above there. Subsequently, the user is able to read the result of the antibody reaction on the basis of the color change of the test strip 5 in the region of the second 3, 3′. Thus, the test system provided according to the invention thus allows in an easily manageable manner the simultaneous examination of a liquid sample with respect to two different microorganisms, such as Streptococcus mutans and Lactobacillus sp.

The experiments below describe the inventive principle by way of a test system for the determination of pathogenic microorganisms in the oral cavity. However, it should be understood that the invention is not restricted to such embodiments but can be used in general for the detection of microorganism in liquid samples such as body fluids.

1. Investigation of the Caries Risk by the Detection of S. mutans and Lactobacillus sp.

### 1.1 Production of the Test Strips

Planar materials capable of chromatography with capillary activity, such as membranes and non-wovens, are fixed on a self-adhesive film. All materials are in capillary contact with each other and permit a stream of liquid to pass from one end of the test strip to the other. The assembly of the materials takes place in such a way that, at the inlet aperture into the cassette, suction non-wovens are present which absorb the mobile buffer and/or a sample with a volume of more than 50 μl and liberate it constantly. The suction non-wovens are in contact with the release non-wovens onto which at least one mobilizable antibody conjugate complex is applied in each case. To increase the test sensitivity, the release non-woven can be doped with antigen, i.e., with whole cells of microorganisms, though preferably with cell fragments containing the corresponding epitopes. Cell fragments, for example, cell wall fragments may be prepared according to the method described below under section 1.6. Typically, the sample is applied onto the release non-woven material. Following the release non-woven material, a porous membrane is present on which the formation of the sandwich complex of conjugated antibody antigen and capture antibody takes place. At the end of the membrane, a further non-woven material is fixed in a manner analogous to the suction non-woven, which collects the liquids applied and consequently guarantees the continuous capillary flow of liquid over the entire test strip.

Applying the antibody SWLA1 conjugated with colloidal gold, the antigen for doping, present as whole cells of S. mutans, the capture antibody SWLA1 and the antibody onto the control line is effected as mentioned above in the dispensing process.

### 1.2 Collection of Saliva

Saliva samples were obtained from different patients by first inducing the formation of saliva by chewing a paraffin pellet. One pellet was used per test subject. The saliva was collected in a measuring beaker with a total volume of 30 ml. The saliva collected during the first 30 seconds was discarded and the saliva formed during the subsequent 5 minutes was collected and subsequently used for further investigation.

### 1.3 Conditioning of Saliva

The flow rate of the entire saliva on the test strip can be unified by a suitable chromatographic buffer. Typically, the buffer reaches the non-woven collecting material within one minute. The antigen-antibody reaction and/or the formation of the sandwich complex, however, take place in the entire phase of the chromatographic process, i.e., for as long as liquid can still flow from the suction to the collecting non-woven. The maximum signal intensity develops within the first 30 minutes following the beginning of chromatography and then remains stable for at least a further 30 minutes.

In order to achieve a better accessibility of the bacteria of the oral flora present in the sample, and to intensify the test signal in this way, different additions can be made to the mobile buffer. For this purpose, investigations into saliva conditioning were carried out in which 200 ml of saliva were carefully mixed in reaction vessels of 1.5 ml with 20 μl of test solution by turning them four times, and incubated for 5 min at room temperature in order to simulate the test conditions on the test strips with respect to the incubation period and the temperature. Subsequently, chromatography was carried out on test strips and the intensity of the test signal as well as the rate of chromatography was assessed.

Different substances described above were examined with respect to their ability to improve the intensity of the signal. It was possible to show that, in the case of labelling with colloidal gold, chaotropic reagents and reagents increasing the ionic strength of the buffer, in particular, such as potassium iodide (0.001-2.0 M) and sodium sulphate (0.001-2.5 M) as well as lysozyme (0.001-20 g/l), papain (0.001-50 g/l) and glutathion (0.001-100 g/l) clearly increase the intensity of the test signal. The skilled person will be in a position, based on the present description, to effect corresponding optimisations for each test system directed to a specific microorganism.

1.4 Applying the Samples onto the Test Device

15 μl of the saliva samples obtained were applied onto the test strip according to the invention by means of pipette. As a result of the high viscosity of saliva, the chromatographic separation is started by the addition of a mobile solvent. A buffer of the following composition is preferably used in the present case as mobile solvent: 20 mM TRIS, 0.5% bovine serum albumin, 0.5% sucrose, 0.7% sodium dodecyl sulphate, 0.5% Tween 80, 1% sodium sulphate, 0.3% sodium cholate, 0.095% sodium azide, 0.05% ProClin 300. The pH is adjusted with hydrochloric acid to a value of 9.0. The buffer serving as mobile solvent is transferred into a reservoir of the test device. Subsequently, the ends of the test strips protruding from the test device are immersed into buffer for at least 15 s (maximum 120 s) as a result of which chromatography is started.

### 1.5 Evaluation of the Signal Intensity

The result can be read within 60 min, preferably 5-30 min, by comparison with a reference standard. In the present case, an optical standard which was obtained with defined concentrations of S. mutans, was used as reference standard.

All numbers expressing quantities or parameters used in the specification are to be understood as additionally being modified in all instances by the term “about”. Notwithstanding that the numerical ranges and parameters set forth, the broad scope of the subject matter presented herein are approximations, the numerical values set forth are indicated as precisely as possible. For example, any numerical value may inherently contains certain errors, evidenced by the standard deviation associated with their respective measurement techniques, or round-off errors and inaccuracies.

1.6 Preparation of Cell Wall Fragments of S. mutans

A 10 ml aliquot of a liquid culture of S. mutans which had been cultured for 24 hours was used for the preparation of S. Mutans cell wall fragments. 1% SDS was added to the aliquot, and the aliquot was centrifuged at 10000×g for 8 minutes. After discarding the supernatant, the pellet was resuspended in 5 ml PBS. Subsequently, 0.25 g Cellytic Express (Sigma-Aldrich) were added and the solution was incubated at 37° C. for 30 minutes. After centrifugation at 5000×g for 1 min and discarding the supernatant, the pellet was resuspended in 1 ml PBS. The resulting solution was dialyzed twice against 2 l PBS for 20 hours using a dialysis membrane with a 100 kDa cut-off. After dialysis, the cell wall fragments dissolved in PBS were transferred in a reaction tube and stored at −80° C. In a last step, the fragments were lyophilized. The cell wall fragments prepared by this method may be resuspended in deionized water or in another suitable solvent upon use, and they may be directly applied to the test strip.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. A test strip for the quantitative or semi-quantitative detection of a microorganism in a liquid sample, comprising:
a first area for accepting the liquid sample, the first area comprising a labelled antibody which is capable of reacting with the microorganism to be detected by forming an immunocomplex; and
a second area spaced from the first area, the second area comprising a capture antibody which is capable of reacting with the microorganism to be detected by forming an immunocomplex and which is immobilised on the test strip;
wherein the test strip is designed in a manner such that the labelled antibody, on contact of the first area of the test strip with a mobile solvent moves into the second area of the test strip such that in the presence of the microorganism to be detected in the liquid sample which is applied in the first area, an immunocomplex is formed from the labelled antibody, the capture antibody and the microorganism, the immunocomplex can be detected by the labelling of the labelled antibody in the second area of the test strip, and wherein the intensity of the signal provided by the labelling provides information on the concentration of the microorganism in the liquid sample.

2. The test strip according to claim 1, wherein the labelled antibody and/or the capture antibody is a monoclonal antibody.

3. The test strip according to claim 1, wherein the labelled antibody is conjugated to particles.

4. The test strip according to claim 3, wherein particles comprise or consist of colloidal gold or colloidal silver.

5. The test strip according to claim 1, wherein the labelled antibody and the capture antibody are directed against a pathogenic bacterium.

6. The test strip according to claim 5, wherein the pathogenic bacterium is a bacterium occurring in the oral cavity.

7. The test strip according to claim 6, wherein the pathogenic bacterium is selected from the group of bacteria of the genus Streptococcus, Lactobacillus, Actinomyces, Prevotella, Actinobacillus, Porphyromonas, Tannerella, Treponema, Fusobacterium, Enterococcus or Helicobacter.

8. The test strip according to claim 7, wherein the pathogenic bacterium is selected from the group of Streptococcus mutans, Streptococcus sobrinus, Streptococcus sanguis, Streptococcus gordonii, Streptococcus mitis, Lactobacillus acidophilus, Lactobacillus gasseri, Lactobacillus casei, Lactobacillus rhamnosus, Actinobacillus actinomycetemcomitans, Porphyromonas gingivalis, Tannerella forsythensis, Actinomyces naeslundii, Treponema denticola, Fusobacterium nucleatum, Prevotella intermedia, Enterococcus faecalis, Treponema denticola, or Helicobacter pylori.

9. The test strip according to claim 1, wherein the labelled antibody and the capture antibody are directed to a pathogenic yeast.

10. The test strip according to claim 9, wherein the yeast is selected from a group of yeasts of the genus Candida, Cryptococcus, Malassecia or Blastoschizomyces.

11. The test strip according to claim 10, wherein the yeast is selected from Candida albicans, Candida krusei, Candida tropicalis, Candida glabrata, Candida lusitaniae, Candida parapsilosis, Cryptococcus neoformans, or Blastoschizomyces capitatus.

12. The test strip according to claim 1, wherein the first area is doped with the microorganism to be detected or with immunologically active parts thereof.

13. The test strip according to claim 12, wherein the first area comprises 90% of the quantity required for the detection of the microorganism to be detected.

14. A method for the quantitative or semi-quantitative detection of a microorganism in a liquid sample, comprising
applying the liquid sample onto the test strip of claim 1,
contacting the test strip with a mobile solvent,
incubating the test strip under conditions in which the labelled antibody moves, on contact of the first area of the test strip with a mobile solvent, into the second area of the test strip, and
detecting the signal in the second area of the test strip;
wherein the intensity of the signal provided by the labelling provides information on the concentration of the microorganism in the liquid sample.

15. The method according to claim 14, wherein the sample is a saliva sample, and wherein Streptococcus mutans and Lactobacillus sp. are detected simultaneously using two test strips.

16. The method according to claim 15, wherein the labelled antibody and/or the capture antibody on the first test strip is SWLA1, and the labelled antibody and/or the capture antibody on the second test strip is SWLA5.

17. A kit for quantitative or semi-quantitative detection of a microorganism, the kit comprising at least one test strip according to claim 1.

18. The kit according to claim 17, further comprising buffers, labelling reagents and/or detection reagents for detecting the labelled antibody.

19. A test system comprising two test strips according to claim 1 and a housing comprising two pairs of apertures, each pair comprising a first aperture and a second aperture spaced from the first aperture, and an inlet aperture for the two test strips, wherein the inlet aperture and the two pairs of first aperture and second aperture are arranged such that the two test strips can be inserted through the inlet aperture into the housing in such a manner that in each case two spaced apart areas of the test strips are accessible through the first apertures and the second apertures.

20. The test system according to claim 19, wherein the areas of the test strips which are accessible through the second apertures comprise the capture antibody.

21. The test system according to claim 20, wherein the areas of the test strips which are accessible through the first apertures comprise the labelled antibody.

22. A method for the determination of the risk of cariogenesis comprising:
applying a liquid sample to a test strip constructed according to claim 1, and analyzing the signal for the presence and/or concentration of a microorganism present in the sample that is indicative of the risk of cariogenesis.
